(19) United States
(12) Patent Application Publication
(10) Pub. No.: US 2026/0165799 A1
(43) Pub. Date: Jun. 18, 2026

(54) ROBOTIC SURGICAL SYSTEM AND METHOD OF SYNCHRONIZING GRASPER WITH SURGICAL INSTRUMENT

(71) Applicant: Cilag GmbH International, Zug (CH)

(72) Inventors: Shinnosuke Inoue, Daly City, CA (US); Mario F. Luces Rosado, Oakland, CA (US)

(21) Appl. No.: 18/985,692

(22) Filed: Dec. 18, 2024

(51) Int. Cl. A61B 34/35; A61B 17/29; A61B 34/00

(52) U.S. Cl. CPC A61B 34/35; A61B 17/29; A61B 34/25

(57) ABSTRACT

A robotic surgical system, comprising: a robotic grasper having a first jaw movably secured to a second jaw; a user interface device (UID) comprising a first finger pad and an opposing second finger pad; and; one or more processors communicatively coupled to the UID and the robotic grasper and configured to control operation of the robotic grasper while a teleoperation mode is engaged, prevent the UID from controlling the robotic grasper in a non-teleoperation mode, and perform the following operations while the system is in the non-teleoperation mode: receive a synchronization sequence including: a first closing action on the first and second finger pads, and an opening action on the first and second finger pads occurring after the first closing action; and determine that, as a result of the synchronization sequence, that the UID is now in a matched relationship with the first and second jaws of the robotic grasper; and then transition the robotic surgical system into the teleoperation mode.

### BACKGROUND

In a surgical robotic system, a robotic arm that has a surgical tool attached to its distal end is remotely operated by a surgeon—this is also referred to as teleoperation. Applications include endoscopic surgery, which involves looking into a patient's body and performing surgery inside, for example the abdominal cavity, using endoscopes and other surgical tools that are attached to the ends of several robotic arms. The system gives the surgeon a close-up view of the surgery site while at the same enabling the surgeon to remotely operate the tool that is attached to the arm by manipulating a handheld user interface device (UID), all in real-time. The tool may be a grasper with jaws, a cutter, a video camera, or an energy emitter such as a laser used for coagulation. The tool is thus controlled by the system, in a precise manner and with high dexterity, in accordance with the surgeon manipulating a handheld UID.

The core function of a UID is to act as an input device to control the translation, rotation, and other actions such as grasping, of end effectors of surgical tools. One type of tool is the grasper, which has two jaws controlled by the open and close movements of a UID's grip linkage or deformable surface that can be squeezed or pressed to close the jaws. Other UIDs have other types of linkages or mechanisms. However, some graspers also have the ability to do more than just grasp tissue or the like. Some graspers also have the ability to fire an implement when the jaws are in a closed configuration. Such implements may include clips and/or staples. However, these graspers have an issue of firing earlier than desired based on activation of the UID with the tool. Therefore, there is a need to allow activation of a UID with a tool without unintendingly firing the tool prior to when the surgeon is ready.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a pictorial view of an example surgical robotic system in an operating arena;

FIG. 2 is a perspective view of a handheld user input device useable within the surgical robotic system of FIG. 1;

FIG. 3 is a perspective view of a user’s hand engaged with the handheld user input device of FIG. 2;

FIG. 4 is a rear elevation view of a user’s hand engaged with the handheld user input device of FIG. 2;

FIG. 5A depicts a top section view of the handheld user input device of FIG. 2 taken along a centerline thereof in an unaffected state;

FIG. 5B depicts a side view of a clip-firing end-effector that is synchronized with the handheld user input device of FIG. 5A;

FIG. 6A depicts a top section view of the handheld user input device of FIG. 2 taken along a centerline thereof in an unaffected state prior to being directed towards a closed state;

FIG. 6B depicts a side view of the clip-firing end-effector of FIG. 5B that is synchronized with the handheld user input device of FIG. 6A;

FIG. 7A depicts a top section view of the handheld user input device of FIG. 2 taken along a centerline thereof in an unaffected state prior to being directed towards an opened state;

FIG. 7B depicts a side view of the clip-firing end-effector of FIG. 5B that is synchronized with the handheld user input device of FIG. 7A.

### DETAILED DESCRIPTION

The following description of certain examples of the technology should not be used to limit its scope. Other examples, features, aspects, versions, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. OVERVIEW OF AN EXAMPLE ROBOTIC SURGICAL SYSTEM

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various versions will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

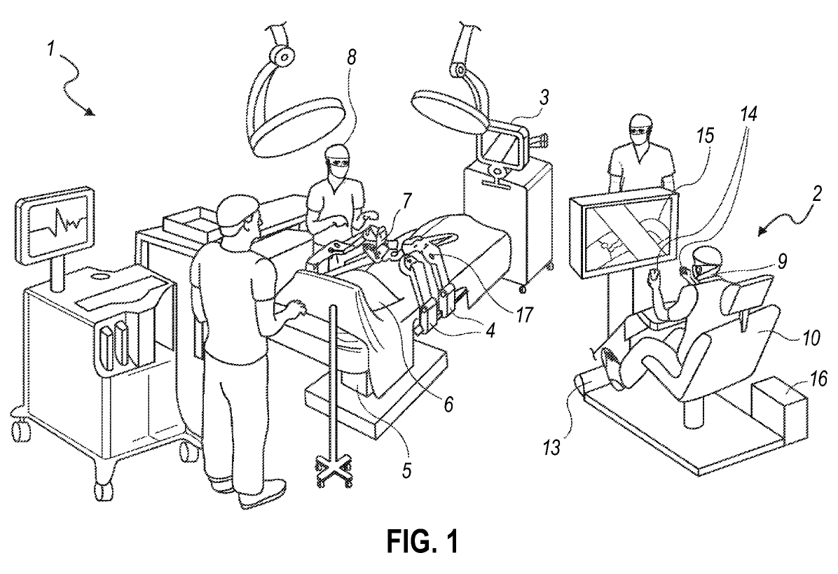

A pictorial view of an example surgical robotic system (1) in an operating arena is shown in FIG. 1. Robotic system (1) includes a user console (2), a control tower (3), and one or more surgical robotic arms (4) at a surgical robotic platform (5), e.g., a table, a bed, etc. Robotic system (1) can incorporate any number of devices, tools, or accessories used to perform surgery on a patient (6). For example, robotic system (1) may include one or more surgical tools (7) used to perform surgery. In one or more versions, surgical tool (7) may be an end effector that is attached to a distal end of surgical arm (4), for executing a surgical procedure.

Each surgical tool (7) may be manipulated manually, robotically, or both, during the surgery. For example, surgical tool (7) may be a tool used to enter, view, or manipulate an internal anatomy of patient (6). In a version, surgical tool (7) may be a grasper that can grasp tissue of the patient, and then fire an implement, such as a clip, as needed. Surgical tool (7) may be controlled manually, by a bedside operator (8); or it may be controlled robotically, via actuated movement of surgical robotic arm (4) to which surgical tool (7) is attached. Robotic arms (4) are shown as a table-mounted system, but in other configurations robotic arms (4) may be mounted in a cart, ceiling, or sidewall, or in another suitable structural support.

Generally, a remote operator (9), such as a surgeon, may use user console (2) to remotely manipulate robotic arms (4) and/or attached surgical tools (7), e.g., teleoperation. User console (2) may be located in the same operating room as the rest of system (1), as shown in FIG. 1. In other environments however, user console (2) may be located in an adjacent or nearby room, or it may be at a remote location, e.g., in a different building, city, or country. User console (2) may comprise a seat (10), foot-operated controls (13), one or more handheld user input devices, “UID” (14), and at least one user display (15) configured to display, for example, a view of the surgical site inside patient (6). As shown in FIG. 1, remote operator (9) is sitting in seat (10) and viewing user display (15) while manipulating foot-operated control (13) and UID (14) in order to remotely control robotic arms (4) and surgical tools (7) (that are mounted on the distal ends of robotic arms (4)).

In some versions, UID (14) may be a portable handheld user input device or controller that is ungrounded with respect to another component of surgical robotic system (1). For example, UID (14) may be ungrounded while either tethered or untethered from user console (2). The term “ungrounded” is intended to refer to implementations where, for example, one or more UIDs (14) are neither mechanically nor kinematically constrained with respect to user console (2). For example, a user may hold UID (14) in a hand and move freely to any possible position and orientation within space only limited by, for example, a tracking mechanism of user console (2). In contrast, a grounded UID (14) can hold its position/orientation/gripping.

In some variations, bedside operator (8) may also operate system (1) in an “over the bed” mode, in which beside operator (8) may be at a side of patient (6) and may simultaneously manipulate robotically-driven tool (7) (such as an end effector attached to arm (4)), e.g., with handheld UID (14) held in one hand, and a manual laparoscopic tool. For example, the left hand of bedside operator (8) may be manipulating handheld UID (14) to control a robotic component, while the right hand of bedside operator (8) may be manipulating a manual laparoscopic tool. Thus, in these variations, bedside operator (8) may perform both robotic-assisted minimally invasive surgery and manual laparoscopic surgery on patient (6).

During an example procedure (surgery), patient (6) may be prepped and draped in a sterile fashion to achieve anesthesia. Initial access to the surgical site may be performed manually while arms (4) of robotic system (1) are in a stowed configuration or withdrawn configuration (to facilitate access to the surgical site). Once access is completed, initial positioning or preparation of robotic system (1) including arms (4) may be performed. Next, the surgery may proceed with remote operator (9) at user console (2) utilizing foot-operated controls (13) and UIDs (14) to manipulate the various end effectors and perhaps an imaging system, to perform the surgery. Manual assistance may also be provided at the procedure bed or table, by sterile-gowned bedside personnel, e.g., bedside operator (8) who may perform tasks such as retracting tissues, performing manual repositioning, and tool exchange upon one or more of robotic arms (4). Non-sterile personnel may also be present to assist remote operator (9) at user console (2). When the procedure or surgery is completed, aspects of robotic system (1) such as user console (2) may be configured or set in a state to facilitate post-operative procedures such as cleaning or sterilization and healthcare record entry or printout via user console (2).

In one version, remote operator (9) holds and moves UID (14) to provide an input command to move a robot arm actuator (17) of robotic system (1). UID (14) may be communicatively coupled to the rest of robotic system (1), e.g., via a console computer system (16). UID (14) can generate spatial state signals corresponding to movement of UID (14), e.g., position and orientation of the handheld housing of UID (14), and the spatial state signals may be input signals to control a motion of robot arm actuator (17). Robotic system (1) may use control signals derived from the spatial state signals, to control proportional motion of robot arm actuator (17). In one version, a console processor of console computer system (16) receives the spatial state signals and generates the corresponding control signals. Based on these control signals, which control how robot arm actuator (17) may be energized to move a segment or link of robotic arm (4), the movement of corresponding surgical tool (7) that is attached to arm (4) may mimic the movement of UID (14). Similarly, interaction between remote operator (9) and UID (14) can generate, for example, a grip control signal that causes a jaw of a grasper of surgical tool (7) to close and grip the tissue of patient (6), while a subsequent grip control signal may cause the grasper to fire a clip into the tissue of patient (6).

Surgical robotic system (1) may include several UIDs (14), where respective control signals are generated for each UID (14) that control the actuators and the surgical tool (7) (end effector) of respective arm (4). For example, remote operator (9) may move a first UID (14) to control the motion of a first actuator (17) that is in a left robotic arm (4), where first actuator (17) responds by moving linkages, gears, etc., in left robotic arm (4). Similarly, movement of a second UID (14) by remote operator (9) controls the motion of another actuator (17), which in turn moves other linkages, gears, etc., of another robotic arm (4). Robotic system (1) may include a right arm (4) that is secured to the bed or table to the right side of patient (6), and a left arm (4) that is at the left side of patient (6). Actuators (17) may include one or more motors that are controlled so that they drive the rotation of a joint of arm (4), to, for example, change, relative to the patient, an orientation of an endoscope or a grasper of surgical tool (7) that is attached to that arm (4). Motion of several actuators (17) in the same arm (4) can be controlled by the spatial state signals generated from a particular UID (14). UIDs (14) can also control motion of respective surgical tool graspers. For example, each UID (14) can generate a respective grip signal to control motion of an actuator, e.g., a linear actuator, which opens or closes jaws of grasper at a distal end of surgical tool (7) to grip tissue within patient (6) and then to subsequently fire a clip into tissue of patient (6).

In some versions, communication between platform (5) and user console (2) may be through control tower (3), which may translate user commands that are received from user console (2) (and more particularly from console computer system (16)) into robotic control commands that transmitted to arms (4) on robotic platform (5). Control tower (3) may also transmit status and feedback from platform (5) back to user console (2). The communication connections between robotic platform (5), user console (2), and control tower (3) may be via wired and/or wireless links, using any suitable ones of a variety of data communication protocols. Any wired connections may be optionally built into the floor and/or walls or ceiling of the operating room. Robotic system (1) may provide video output to one or more displays, including displays within the operating room as well as remote displays that are accessible via the Internet or other networks. The video output or feed may also be encrypted to ensure privacy and all or portions of the video output may be saved to a server or electronic healthcare record system.

One problem to be solved with surgical robotic system (1) is how or when to engage or resume (i.e., re-engage) teleoperation safely, without causing unintended movements of surgical tools (7) that may lead to tissue damage. Without the solution described herein, there is a risk that re-engaging teleoperation, in particular when teleoperation was interrupted for some reason (such as if system (1) entered a disengaged mode), could result in a surgical robotic grasper to suddenly open, suddenly close, clamp down too hard on tissue, release tissue that had been previously grasped, and or unintentional firing of a clip, etc.

### II. Example of a UID Actuator

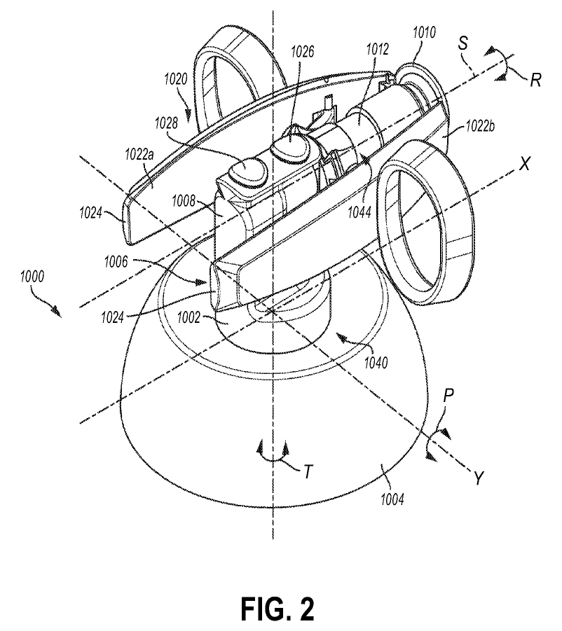

FIG. 2 shows an example of a UID actuator (1020) in a closed stated, which may be part of a UID (1000), which is similar to UID (14) as shown in FIG. 1. UID actuator (1020) includes opposing first and second finger pads (1022a, 1022b) extending from wrist (1010) toward joystick (1008) and a central portion (1002) of a space joint (1006). Opposing first and second finger pads (1022a, 1022b) extend distally beyond space joint (1006). In such instances, wrist (1010) may be proximal to space joint (1006), and distal ends (1024) of opposing first and second finger pads (1022a, 1022b) may be distal to space joint (1006), which mirrors the jaws being positioned distal to the articulation joints of a robotic tool, for example tool (7). Applying an actuation force to opposing first and second finger pads (1022a, 1022b) may comprise an input control for a surgical tool. For example, applying a pinching force to opposing first and second finger pads (1022a, 1022b) can close and/or clamp jaws of an end effector. In various instances, applying a spreading force can open and/or release the jaws of end effector, such as for a spread dissection task, for example. UID actuator (1020) can include at least one sensor for detecting input control motions applied to opposing first and second finger pads (1022a, 1022b). For example, the end effector actuator can include a displacement sensor and/or a rotary encoder for detecting the input control motions applied to pivot opposing first and second finger pads (1022a, 1022b) relative to a shaft (1012). Such sensors can generate grasp signals applied to UID (1000) by the operator that can be translated to jaw angle and grip force to be utilized by an end effector in teleoperation with UID (1000).

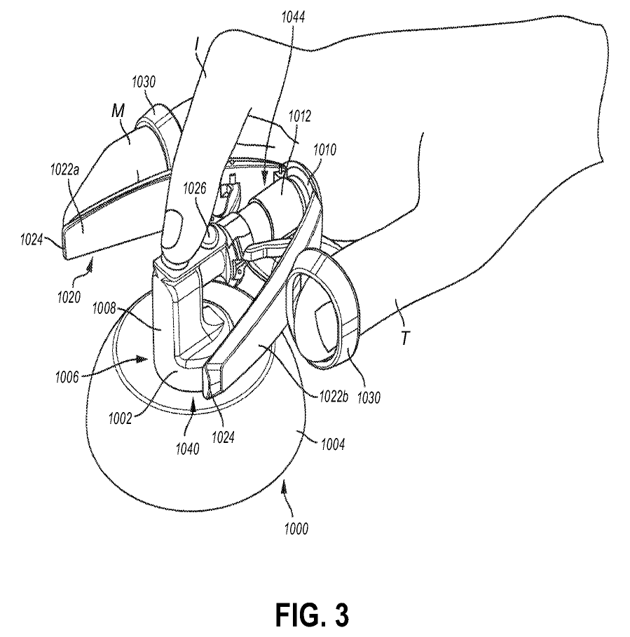

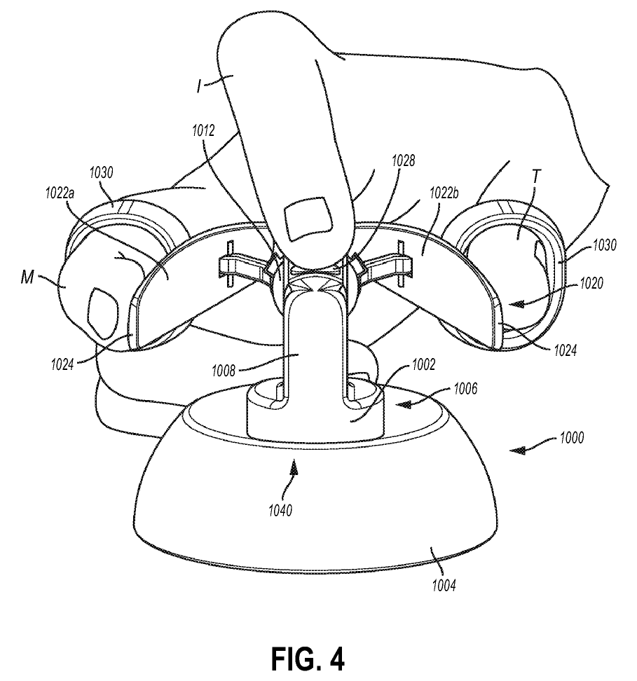

In various versions, UID actuator (1020) can include first and second loops (1030a, 1030b), which are dimensioned and positioned to receive a surgeon's digits. For example, referring primarily to FIGS. 3 and 4, a surgeon's thumb (T) may be positioned through one of loops (1030a or 1030b) and the surgeon's middle finger (M) may be positioned through other loop (1030b or 1030a). In such instances, the surgeon can pinch and/or spread his thumb (T) and middle finger (M) to actuate UID actuator (1020), which is shown in FIGS. 3 and 4 in an opened state. In other versions, first and second loops (1030a, 1030b) can be structured to receive more than one digit and, depending on the placement of first and second loops (1030a, 1030b), different digits may engage first and second loops (1030a, 1030b). In various versions, first and second finger loops (1030a, 1030b) can facilitate spread dissection functions and/or translation of robotic tool (7) upward or downward (i.e., the application of a vertical force at space joint (1006), for example). In certain instances, first and second loops (1030a, 1030b) can define complete loops; however, in other instances, partial loops (e.g., half-circles) can be utilized. In still other instances, UID actuator (1020) may not include first and second loops (1030a, 1030b). For example, UID actuator (1020) can be spring-biased outwardly such that loops are not needed to draw opposing first and second finger pads (1022a, 1022b) apart, such as for spread dissection functions.

Opposing first and second finger pads (1022a, 1022b) of UID actuator (1020) define a line of symmetry that may be aligned with the longitudinal shaft axis (S) along which shaft (1012) extends when opposing first and second finger pads (1022a, 1022b) are in unactuated positions. The line of symmetry may be parallel to the axis (X) through multi-dimensional space joint (1006). Moreover, the central axis of joystick (1008) may be aligned with the line of symmetry. In various versions, motion of opposing first and second finger pads (1022a, 1022b) can be independent. In other words, opposing first and second finger pads (1022a, 1022b) can be displaced asymmetrically relative to longitudinal shaft axis (S) during an actuation. Displacement of opposing first and second finger pads (1022a, 1022b) can depend on the force applied by the surgeon, for example. With certain surgical tools, the jaws of the end effector can pivot about an articulation axis such that various closed positions of the jaws may not be longitudinally aligned with the shaft of the surgical tool. Moreover, in certain instances, it can be desirable to hold one jaw stationary, such as against fragile tissue and/or a critical structure, and to move the other jaw relative to the non-moving jaw. To accommodate such closure motions, the range of motion of opposing first and second finger pads (1022a, 1022b) on UID (1000) can be larger than the range of motion of the jaws of the end effector, for example.

In one or more versions, UID (1000) may also include at least one additional actuator, such as actuation buttons (1026, 1028), for example, which can provide additional controls at the surgeon's fingertips. For example, actuation buttons (1026, 1028) may be positioned on joystick (1008) of UID (1000) such that the surgeon can access actuation buttons (1026, 1028) with a digit, such as an index finger (I). Actuation buttons (1026, 1028) can correspond to buttons for activating a surgical tool, such as firing, extending, activating, translating, and/or retracting a knife, energizing one or more electrodes, adjusting an energy modularity, affecting diagnostics, biopsy sampling, ablation, and/or other surgical tasks, for example. In other instances, actuation buttons (1026, 1028) can provide inputs to an imaging system to adjust a view of the surgical tool, such as zooming in/out, panning, tracking, titling, and/or rotating, for example. In certain instance the actuators can be positioned in different locations than the actuation buttons (1026, 1028), such as positioned for use by a thumb or another digit, for example. Additionally, or alternatively, the actuators can be provided on a touch screen and/or foot pedal, for example.

In one or more versions, UID (1000) may also include a plurality of mechanical joints, which can be elastically-coupled components, sliders, journaled shafts, hinges, and/or rotary bearings, for example. The mechanical joints may include a first joint (1040) (at space joint (1006)), intermediate a base (1004) and central portion (1002), which may allow for rotation and tilting of central portion (1002) relative to base (1004), and a second joint (1044), which may allow for rotation of wrist (1010) relative to joystick (1008). In various versions, six degrees of freedom of a robotic end effector (e.g., three-dimensional translation and rotation about three different axes) can be controlled by user inputs at only these two joints (1040, 1044), for example. With respect to motion at first joint (1040), central portion (1002) can be configured to float relative to base (1004) at elastic couplings. With respect to second joint (1044), wrist (1010) can be rotatably coupled to shaft (1012), such that wrist (1010) can rotate in the direction R (FIG. 2) about the shaft axis (S). Rotation of wrist (1010) relative to shaft (1012) can correspond to a rolling motion of an end effector about a central tool axis. Rotation of wrist (1010) by the surgeon to roll an end effector provides control of the rolling motion at the surgeon's fingertips and corresponds to a first-person perspective control of the end effector (i.e., from the surgeon's perspective, being “positioned” at the jaws of the remotely-positioned end effector at the surgical site). Such placement and perspective can be utilized to supply precision control motions to UID (1000) during portions of a surgical procedure (e.g., a precision motion mode).

### III. Synchronizing UID Actuator with an End Effector

The present system determines when user input from a user interface device (UID) matches a grasper angle or grasper force of a surgical robotic grasper, at which point the surgical robotic grasper goes from a non-teleoperation mode to a teleoperation mode. In one or more versions, non-teleoperation mode includes one of a disengaged mode or a pause mode. In one or more versions, disengaged mode may be when the UID is disengaged from controlling the robotic grasper prior to a teleoperation session and pause mode may be when the UID is disengaged from controlling the robotic grasper to pause the teleoperation session. The UID may not be mechanically constrained to maintain the same position, orientation, and grasper angle or force as the surgical tool. Accordingly, a synchronization sequence is described that, when performed, detects when the grasper part of the UID is matching the surgical tool grasper (with respect to angle and force, i.e., the UID and the surgical robotic grasper are within an acceptable threshold of agreeing with each other) before the system engages or resumes (i.e., re-engages) teleoperation. Two devices that are matched, in this context, means that the positions and orientations or other relevant values of the two devices are within a threshold of agreement with each other. The process of matching means the process of bringing the positions and orientations or other relevant values of the two devices to within a threshold of agreement with each other. Upon detecting this match, the system may respond with an alert to the user and with engaging teleoperation of the surgical robotic grasper.

In one example, to match a UID with an end effector the user is expected to open the UID, past a UID open threshold, or start with the UID in an open position or orientation. For example, the user can set a grip crank of the UID to an angle greater than a UID open threshold. Then the user closes the UID. For example, the user can close the UID past a UID closed threshold. However, some issues can arise in a matching algorithm of this nature. If the end effector is a grasper that can fire an implement as discussed above, and if the act of closing the UID matches with the orientation of the end effector and the systems become matched, this act of closing could fire the implement from the grasper prior to the user placing the grasper in the correct position for firing.

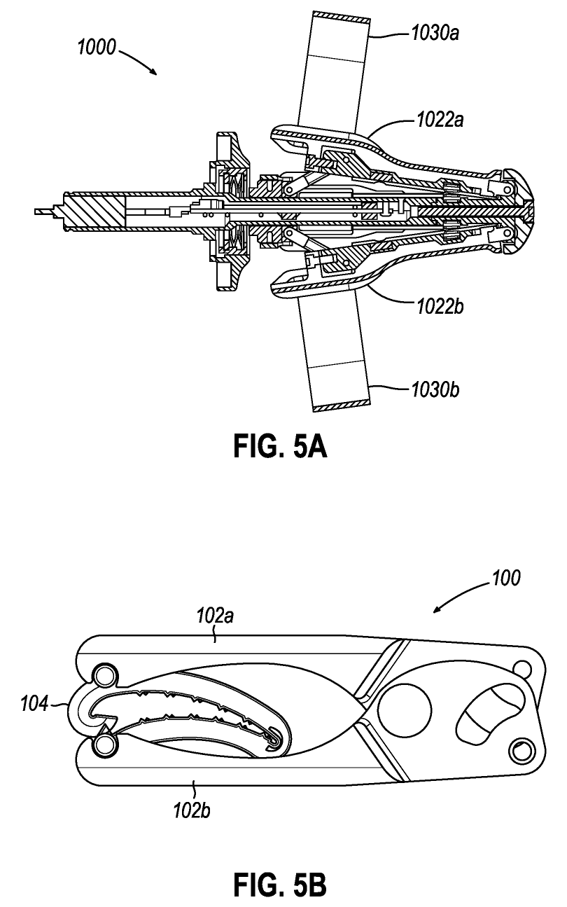

FIGS. 5A, 6A, and 7A show various states of UID (1000) through a synchronization sequence of the present disclosure, while FIGS. 5B, 6B, AND 7B show various states of an end effector (100) through the synchronization sequence of the present disclosure. FIG. 5A shows UID (1000) in an unaffected state, prior to the initial step of the synchronization sequence of the present disclosure. FIG. 5B shows end effector (100) after end effector (100) has been loaded onto a robotic arm and having been inserted to its desired location relative to the patient. In this initial, unaffected state, first and second jaws (102a, 102b) of end effector (100) are partially open such that clip (104) is not able to be fired.

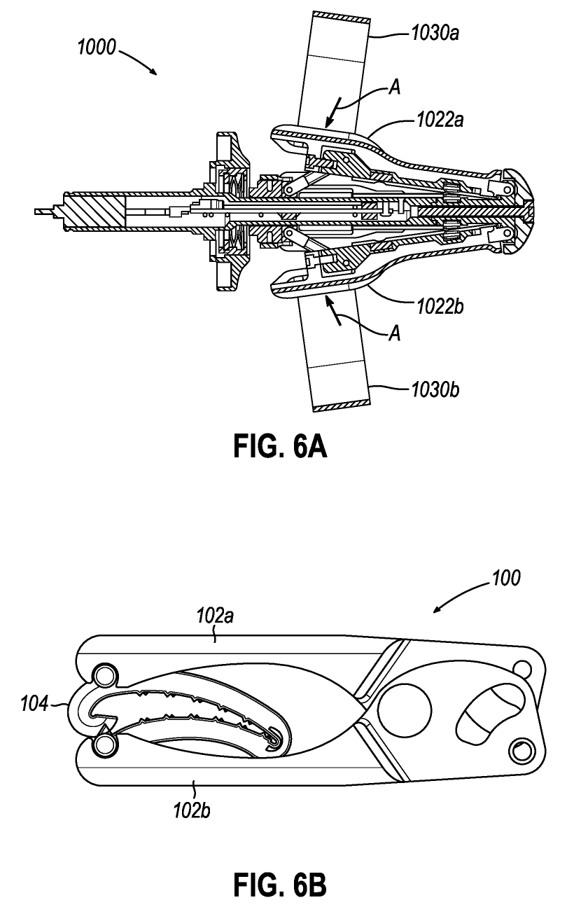

FIG. 6A shows UID (1000) still in an unaffected state prior to being directed towards a closed state such as shown in FIG. 2, which is the first step of the synchronization sequence of the present disclosure. To direct UID (1000) towards the closed state, the operator of UID (1000) will apply pressure to opposing first and second finger pads (1022a, 1022b) to depress opposing first and second finger pads (1022a, 1022b), as shown with arrows (A). FIG. 6B shows end effector (100) in the same position as shown in FIG. 5B because with this first step of the synchronization sequence of the present disclosure, end effector (100) is not yet fully synchronized with UID (1000). End effector (100) must go past a certain closed distance threshold before the operator of UID (1000) may continue on to the next step of the synchronization sequence. However, even in passing the closed distance threshold clip (104) remains positioned within end effector (100) in an inactivated state.

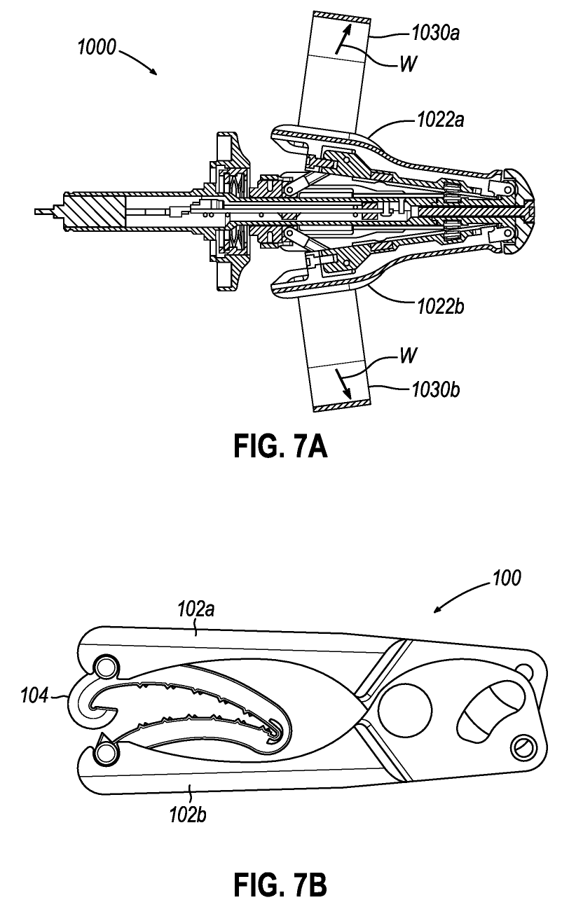

FIG. 7A shows UID (1000) still in an unaffected state prior to being directed towards an opened state such as shown in FIGS. 3 and 4, which is the final step of the synchronization sequence of the present disclosure. To direct UID (1000) towards the opened state, the operator of UID (1000) will apply pressure to first and second loops (1030a, 1030b) as shown with arrows (W), which will cause opposing first and second finger pads (1022a, 1022b) to open. This opening actions on first and second loops (1030a, 1030b) performed by the operator will have to move opposing first and second finger pads (1022a, 1022b) past an opening threshold of UID (1000). FIG. 7B shows end effector (100) with first and second jaws (102a, 102b) in an open configuration, meaning opposing first and second finger pads (1022a, 1022b) were successfully moved past the opening threshold of UID (1000). The operator input to this point now has completed the synchronization sequence with UID (1000) and teleoperation is now engaged. When UID (1000) has finished the synchronization sequence, end effector (100) will start opening and try to match up with the position of UID (1000). Furthermore, when the opening action occurs on opposing first and second finger pads (1022a, 1022b), clip (104) is now in an activated state. If the operator is ready for clip (104) to be fired, the operator may then apply pressure to opposing first and second finger pads (1022a, 1022b) to depress opposing first and second finger pads (1022a, 1022b), in a similar action to what is shown in FIG. 6A, and clip (104) will fire.

The synchronization sequence described above may be monitored by the console processor of console computer system (16). The console processor of console computer system (16) keeps track of a teleoperation mode and an unmatched/matched state, of UID (1000) and end effector (100). The console processor of console computer system (16) outputs an indication of a matched state, when (or in response to) the operator input from UID (1000) matching the angle and/or force of end effector (100). This indication could be presented to the operator in the form of an audio message, such as a tone, sound or speech, a visual alert such as a light turning on or flashing, a text or graphic message displaying on display (15) of user console (2) , a tactile alert such as vibration, for example of UID (1000), an armrest or a seat vibrator in user console (2), or other notification to the operator at user console (2). Or, the indication could trigger the engagement of teleoperation, with or without a further notification, in various embodiments.

### IV. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A robotic surgical system, comprising: (a) a robotic grasper having a first jaw movably secured relative to a second jaw; (b) a user interface device (UID) comprising a first finger pad and an opposing, second finger pad; and (c) one or more processors communicatively coupled to the UID and the robotic grasper and configured to control operation of the robotic grasper in accordance with user input from the UID while a teleoperation mode is engaged, prevent the UID from controlling the robotic grasper while the system is in a non-teleoperation mode, and perform the following operations while the system is in the non-teleoperation mode: (i) receive a synchronization sequence of detected user actions through the UID, wherein the synchronization sequence includes: (A) a first closing action on the first and second finger pads, and (B) an opening action on the first and second finger pads occurring after the first closing action, and (ii) determine that, as a result of the synchronization sequence, that the UID is now in a matched relationship with the first and second jaws of the robotic grasper, and then (iii) transition the robotic surgical system into the teleoperation mode.

### Example 2

The robotic surgical system of Example 1, wherein when the first closing action occurs, the first and second jaws of the robotic grasper remain in a partially opened state.

### Example 3

The robotic surgical system of Example 1, wherein when the opening action occurs after the first closing action, the first and second jaws of the robotic grasper enter into an opened state.

### Example 4

The robotic surgical system of Example 1, wherein the first closing action on the first and second finger pads needs to move past a pre-defined closing threshold in order to meet a first requirement of the synchronization sequence.

### Example 5

The robotic surgical system of Example 1, wherein the opening action on the first and second finger pads needs to move past a pre-defined opening threshold in order to meet a second requirement of the synchronization sequence.

### Example 6

The robotic surgical system of Example 1, the UID further comprising a first loop in a spatial relationship with the first finger pad and a second loop in a spatial relationship with the second finger pad.

### Example 7

The robotic surgical system of Example 6, wherein the opening action on the first and second finger pads occurs by applying an upward pressure to both the first loop and the second loop.

### Example 8

The robotic surgical system of Example 1, wherein the robotic grasper is configured to fire one or more implements.

### Example 9

The robotic surgical system of Example 8, wherein the one or more implements are selected from the group consisting of one or more clips or one or more staples.

### Example 10

The robotic surgical system of Example 8, wherein when the first closing action on the first and second finger pads occurs, the one or more implements remain positioned within the robotic grasper in an inactivated state.

### Example 11

The robotic surgical system of Example 8, wherein when the opening action on the first and second finger pads occurs, the one or more implements enter an activated state.

### Example 12

The robotic surgical system of Example 8, wherein a second closing action on the first and second finger pads occurring during teleoperation mode will fire the one or more implements.

### Example 13

The robotic surgical system of Example 1, wherein the non-teleoperation mode includes one of a disengaged mode or a pause mode.

### Example 14

The robotic surgical system of Example 13, wherein disengaged mode is when the UID is disengaged from controlling the robotic grasper prior to a teleoperation session; and wherein pause mode is when the UID is disengaged from controlling the robotic grasper to pause the teleoperation session.

### Example 15

A method for engaging a teleoperation mode of a surgical robotic system, the robotic surgical system including (a) a robotic grasper having a first jaw movably secured relative to a second jaw, (b) a user interface device (UID) comprising a first finger pad and an, opposing second finger pad, and (c) one or more processors communicatively coupled to the UID and the robotic grasper and configured to control operation of the robotic grasper in accordance with user input from the UID while teleoperation is engaged, the method comprising: (a) placing the robotic grasper into a desired location, wherein each of the first and second jaws is in a partially open position; (b) initiating a synchronization sequence of detected user actions through the UID, wherein the synchronization sequence includes: a first closing action on the first and second finger pads, and an opening action on the first and second finger pads occurring after the first closing action; (c) determining, by the one or more processors, that, as a result of the synchronization sequence, that the UID is now in a matched relationship with the first and second jaws of the robotic grasper; and (d) engaging the robotic surgical system into teleoperation mode.

### Example 16

The method of Example 15, wherein when the first closing action occurs, the first and second jaws of the robotic grasper remain in a partially opened state.

### Example 17

The method of Example 15, wherein when the opening action occurs after the first closing action, the first and second jaws of the robotic grasper enter into an opened state.

### Example 18

The method of Example 15, wherein the robotic grasper is configured to fire one or more implements, such that during the step of providing the robotic surgical system, the one or more implements are positioned within the first and second jaws of the robotic grasper.

### Example 19

The method of Example 18, further comprising the step of initiating a second closing action on the first and second finger pads occurring during the teleoperation mode, wherein the second closing action will fire the one or more implements.

### Example 20

A method for re-engaging a teleoperation mode of a surgical robotic system in a pause mode, the robotic surgical system including (a) a robotic grasper having a first jaw movably secured relative to a second jaw, (b) a user interface device (UID) comprising a first finger pad and an, opposing second finger pad, and (c) one or more processors communicatively coupled to the UID and the robotic grasper and configured to control operation of the robotic grasper in accordance with user input from the UID while teleoperation is engaged, the method comprising: (a) initiating a synchronization sequence of detected user actions through the UID, wherein the synchronization sequence includes: a first closing action on the first and second finger pads, and an opening action on the first and second finger pads occurring after the first closing action; (b) determining, by the one or more processors, that, as a result of the synchronization sequence, that the UID is now in a matched relationship with the first and second jaws of the robotic grasper; and (c) engaging the robotic surgical system into teleoperation mode.

### V. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of uterine manipulator instruments, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A robotic surgical system, comprising:
(a) a robotic grasper having a first jaw movably secured relative to a second jaw;
(b) a user interface device (UID) comprising a first finger pad and an opposing, second finger pad; and
(c) one or more processors communicatively coupled to the UID and the robotic grasper and configured to control operation of the robotic grasper in accordance with user input from the UID while a teleoperation mode is engaged, prevent the UID from controlling the robotic grasper while the system is in a non-teleoperation mode, and perform the following operations while the system is in the non-teleoperation mode:
(i) receive a synchronization sequence of detected user actions through the UID, wherein the synchronization sequence includes:
(A) a first closing action on the first and second finger pads, and
(B) an opening action on the first and second finger pads occurring after the first closing action, and
(ii) determine that, as a result of the synchronization sequence, that the UID is now in a matched relationship with the first and second jaws of the robotic grasper, and then
(iii) transition the robotic surgical system into the teleoperation mode.

2. The robotic surgical system of claim 1, wherein when the first closing action occurs, the first and second jaws of the robotic grasper remain in a partially opened state.

3. The robotic surgical system of claim 1, wherein when the opening action occurs after the first closing action, the first and second jaws of the robotic grasper enter into an opened state.

4. The robotic surgical system of claim 1, wherein the first closing action on the first and second finger pads needs to move past a pre-defined closing threshold in order to meet a first requirement of the synchronization sequence.

5. The robotic surgical system of claim 1, wherein the opening action on the first and second finger pads needs to move past a pre-defined opening threshold in order to meet a second requirement of the synchronization sequence.

6. The robotic surgical system of claim 1, the UID further comprising a first loop in a spatial relationship with the first finger pad and a second loop in a spatial relationship with the second finger pad.

7. The robotic surgical system of claim 6, wherein the opening action on the first and second finger pads occurs by applying an upward pressure to both the first loop and the second loop.

8. The robotic surgical system of claim 1, wherein the robotic grasper is configured to fire one or more implements.

9. The robotic surgical system of claim 8, wherein the one or more implements are selected from the group consisting of one or more clips or one or more staples.

10. The robotic surgical system of claim 8, wherein when the first closing action on the first and second finger pads occurs, the one or more implements remain positioned within the robotic grasper in an inactivated state.

11. The robotic surgical system of claim 8, wherein when the opening action on the first and second finger pads occurs, the one or more implements enter an activated state.

12. The robotic surgical system of claim 8, wherein a second closing action on the first and second finger pads occurring during teleoperation mode will fire the one or more implements.

13. The robotic surgical system of claim 1, wherein the non-teleoperation mode includes one of a disengaged mode or a pause mode.

14. The robotic surgical system of claim 13, wherein disengaged mode is when the UID is disengaged from controlling the robotic grasper prior to a teleoperation session; and wherein pause mode is when the UID is disengaged from controlling the robotic grasper to pause the teleoperation session.

15. A method for engaging a teleoperation mode of a surgical robotic system, the robotic surgical system including (a) a robotic grasper having a first jaw movably secured relative to a second jaw, (b) a user interface device (UID) comprising a first finger pad and an, opposing second finger pad, and (c) one or more processors communicatively coupled to the UID and the robotic grasper and configured to control operation of the robotic grasper in accordance with user input from the UID while teleoperation is engaged, the method comprising:
(a) placing the robotic grasper into a desired location, wherein each of the first and second jaws is in a partially open position;
(b) initiating a synchronization sequence of detected user actions through the UID, wherein the synchronization sequence includes:
(i) a first closing action on the first and second finger pads, and
(ii) an opening action on the first and second finger pads occurring after the first closing action;
(c) determining, by the one or more processors, that, as a result of the synchronization sequence, that the UID is now in a matched relationship with the first and second jaws of the robotic grasper; and
(d) engaging the robotic surgical system into teleoperation mode.

16. The method of claim 15, wherein when the first closing action occurs, the first and second jaws of the robotic grasper remain in a partially opened state.

17. The method of claim 15, wherein when the opening action occurs after the first closing action, the first and second jaws of the robotic grasper enter into an opened state.

18. The method of claim 15, wherein the robotic grasper is configured to fire one or more implements, such that during the step of providing the robotic surgical system, the one or more implements are positioned within the first and second jaws of the robotic grasper.

19. The method of claim 18, further comprising the step of initiating a second closing action on the first and second finger pads occurring during the teleoperation mode, wherein the second closing action will fire the one or more implements.

20. A method for re-engaging a teleoperation mode of a surgical robotic system in a pause mode, the robotic surgical system including (a) a robotic grasper having a first jaw movably secured relative to a second jaw, (b) a user interface device (UID) comprising a first finger pad and an, opposing second finger pad, and (c) one or more processors communicatively coupled to the UID and the robotic grasper and configured to control operation of the robotic grasper in accordance with user input from the UID while teleoperation is engaged, the method comprising:
(a) initiating a synchronization sequence of detected user actions through the UID, wherein the synchronization sequence includes:
(i) a first closing action on the first and second finger pads, and
(ii) an opening action on the first and second finger pads occurring after the first closing action;
(b) determining, by the one or more processors, that, as a result of the synchronization sequence, that the UID is now in a matched relationship with the first and second jaws of the robotic grasper; and
(c) engaging the robotic surgical system into teleoperation mode.
